(19) United States
(12) Patent Application Publication
(10) Pub. No.: US 2007/0275108 A1
(43) Pub. Date: Nov. 29, 2007

(54) Life Span Managment

(76) Inventor: Bard J. Geesamen, Cambridge, MA (US)

Correspondence Address: FISH & RICHARDSON PC, P.O. BOX 1022, MINNEAPOLIS, MN, 55440-1022, US

(21) Appl. No.: 10/583,187

(22) PCT Filed: Dec. 20, 2004
(86) PCT No.: PCT/US04/42907

§ 371 Date: Jun. 14, 2007

(30) Foreign Application Priority Data
Dec. 19, 2003 (US) 60531507

(51) Int. Cl. A61K 36/8962; A61K 31/352; A61K 47/00; G06G 7/48; C12Q 1/02; A61K 31/455

(52) U.S. Cl. 424/754; 435/29; 514/356; 514/456; 514/789; 703/11

(57) ABSTRACT

Natural products can confer protective effects, effects identical to or similar to protective effects of a “protective” genetic variant. Through the administration (e.g. consumption, ingestion, topical application, etc) of natural products, it is possible in some cases to counter one or more effects of a detrimental gene and mimic one or more effects of a protective gene,

### CROSS-REFERENCE TO RELATED APPLICATIONS

This application claims priority to U.S. Patent Application No. 60/531,507, filed on Dec. 19, 2003, the entire contents of which are hereby incorporated by reference. This application also incorporates by reference PCT/US03/15370 and U.S. patent application Ser. No. 10/219,443, in their entireties.

### TECHNICAL FIELD

This invention relates to lifespan and health management.

### BACKGROUND

Approximately 50% of the variance in human lifespan is genetic. By genetically comparing the DNA of long lived individuals (e.g. centenarians) with that of younger individuals it is possible to identify the gene variants that contribute to the variance in human lifespan. An example of a genetic variant is the longevity-associated variant of the microsomal triglyceride transfer protein (MTP) gene (see, e.g., PCT/US03/15370). One detrimental variant is a promoter SNP that upregulates MTP expression levels.

### SUMMARY

This disclosure includes methods and compositions for evaluating the effects of natural products on biological parameters associated with longevity.

In one aspect, this disclosure features a method of providing an agent to a subject that has a particular allele of a gene, e.g., a gene associated with altered cholesterol regulation, e.g., an MTP gene. For example, the subject has a detrimental allele of the gene relative to a longevity associated allele. The term “detrimental allele” refers to an allele that is associated with reduced longevity or quality than at least one other allele of the same gene and/or position. PCT/US03/15370 describes alleles of the MTP gene. If a detrimental allele is present, then the subject can be provided with a recommendation to consume a nutraceutical or natural product. Extracts of garlic and citric flavanoids, for example, are two naturally occurring products that decrease MTP expression levels and thereby offset the impact of the detrimental variant. Accordingly, for a subject that has the detrimental variant, the method includes providing the subject with instructions to consume garlic and citric flavanoids, or extracts thereof. In one embodiment, the method includes administering garlic and citric flavanoids, or extracts thereof. Other nutraceuticals which decrease MTP-expression levels can also be used.

In another example, niacin is used for individuals that have a detrimental allele of the CETP gene.

It is also possible to develop genetic tests that can be used to identify an individual's genetic profile. Based on this profile, it is possible to recommend natural products to optimize an individual's health and increase his chances. of living a long life. One example of this is a direct to consumer test where a cheek swab is performed and the sample is sent to a central facility for analysis and a set of nutritional recommendations are made.

In one aspect, the disclosure features a method that includes:

1) genetically comparing a subjects genetic composition (e.g., by analyzing DNA, RNA, or protein) and other blood metabolic markers with a database that includes information about protected or potentially protected individuals. Protected and potentially protected individuals include, e.g., centenarians and long-lived individuals (e.g., individuals living to at least the 65, 75, 80, 85, 90, 95, or 98^{th} percentile of the population);

2) characterizing a subject (e.g., health and longevity) by comparison of parameters to corresponding parameters long-lived individuals and centenarians, e.g., using profiles; and

3) making nutraceutical/nutritional/dietary recommendations to optimize health and longevity based on results of the comparison.

In one embodiment, a profile of parameters from a subject is compared to one or more profiles from a database of long-lived individuals or centenarians. The profile in the database can be the profile of one of the long-lived individuals or centenarians, or it can be a derivative profile, e.g. a profile that a function of a plurality of individual profiles, e.g., an average, consensus, median, mode, etc. The profile may include a range associated with each parameter of the profile. As used herein, a profile is a characterization that encompasses one or more parameters, e.g., qualitative or quantitative information about one or more parameters, e.g., a range, tolerance, bin, etc.

For example, the plurality of individual profiles can represent a subset of individuals, e.g., a subset of long-lived individuals or centenarians. For example, the subset can be identified by clustering individuals, e.g., to identify groups of individuals with a similar profile.

A parameter can refer to a biological component or a property thereof, e.g., a gene, a nucleotide position within a gene (e.g., in a coding or non coding region), a mRNA form (e.g., a splice variant), a protein, a protein modification, protein localization, etc. The parameter can be qualitative or quantitative, or both. For example, a nucleotide at a particular position can be homozygous or heterozygous. A gene can be expressed or not expressed, or can be expressed at a particular level, and so on. In one embodiment, the parameter relates to a genetic parameter.

Profiles from the database can be compared to the subject's profile, e.g., using established methods for multivariate analysis. For example, a distance function can be used to compare the subjects' profile to one or more profiles from the database, e.g., to identify one or more matches.

In one aspect, the invention features a database that includes a plurality of records. Each record can include an association with a profile and an association with a nutraceutical. The database can be access, e.g., using a profile from a subject. The database can include a filter that returns records or information from records for profiles that are related to (e.g., “match”), the subject's profile. The information from such records can be delivered, e.g., displayed, transmitted, or stored. The method can include accessing the returned information to provide instructions to the subject to administer the nutraceutical. Where the information is delivered directly to the subject, the subject can administer the relevant nutraceutical. Information can be delivered (e.g., transmitted and/or received) across a network (e.g., intranet or Internet), e.g., in public or secure form. For example, the information can be made accessible through a web site or other portal, the website can be secure.

The method can further include monitoring the subject, e.g., before, during, or after administering the nutraceutical.

In another aspect, the invention features a method of identifying an association between a composition (e.g., a nutraceutical, a test compound, a diet or dietary substance) and biological parameter. The method includes contacting the composition to a test cell or administering the composition to a test organism. The test cell or test organism is evaluated, e.g., to determine a profile. The profile can be compared to profiles obtained from long-lived individuals or centenarians or from cells or organisms (e.g., transgenic organisms) that include one or more gene derived from long-lived individuals or centenarians, or other genetic modification to confer a lifespan trait of the long-lived individuals or centenarians. If the profiles are matched (e.g., are within a threshold or satisfy a criterion), then the composition can be associated with the test profile. The profile can include one or more parameters, e.g., a parameter that relates to MTP or CETP gene expression or protein activity. In one embodiment, the test cell is derived from a subject that does not have a lifespan-extending trait, e.g., a subject having a detrimental allele of a gene, e.g., MTP or CETP.

In one embodiment, the test cell is derived from a subject that does not have a particular genetic modification that confers a lifespan trait of the long-lived individuals or centenarians. For example, if the lifespan trait is the MTP locus, the individuals have the detrimental MTP allele.

In another aspect, the disclosure features a method of providing a recommendation for consuming a nutraceutical. The method includes evaluating a subject for biological parameters, comparing parameters to database of protected individuals, characterizing subject by comparison to database, and making recommendations based on the results. Exemplary parameters include a parameter about a gene or gene product, e.g., mRNA, protein expression, levels, protein modification or protein localization. Other exemplary parameters include a metabolic marker, e.g. a blood marker, e.g., a cholesterol level, e.g., HDL or LDL level. In one embodiment, the comparison is made using a profile, e.g., a profile from a long-lived individual or a profile based on a plurality of individual profiles (e.g., an average or consensus profile). In one embodiment, the profile is a subset identified by clustering. In one embodiment, the subject is monitored before, during, or after administering the nutraceutical

The details of one or more embodiments of the invention are set forth in the description below. Other features, objects, and advantages of the invention will be apparent from the description and from the claims.

### DETAILED DESCRIPTION

The inventors have discovered, inter alia, that certain natural products can confer protective effects, effects identical to or similar to protective effects of a “protective” genetic variant. Through the administration (e.g. consumption, ingestion, topical application, etc) of natural products, it is possible in some cases to counter one or more effects of a detrimental gene and mimic one or more effects of a protective gene, thereby positively impacting an individual's longevity or longevity profile. In particular this can be done on an individual basis, e.g., so that the individual selects one or more (e.g., an optimized combination) of natural products for administration.

Natural Products

Many natural products, whole foods, food ingredients, and supplements are known to have specific health or medical benefits. Administration of these products, typically orally, can improve general health and can be useful in affecting longevity.
Exemplary natural products include: Dietary supplements
Vitamins—A (beta-carotene or retinol), D (calciferols), E (tocopherols), K (phylloquinone), B-1 (thiamine), B-2 (riboflavin), B-6 (pyridoxine), B-12 (cobalamin), C (ascorbic acid), Biotin, Choline, folic acid (folate, B-vitamin), niacin (sometimes called vitamin B-3), pantothenic acid
Antioxidants (a variety of molecules including vitamins E, C, A and trace elements such as selenium, copper and zinc)
Minerals—calcium, iodine, iron, copper, chromium, magnesium, manganese, molybdenum, zinc, potassium, selenium, phosphorus, boron, fluorine, germanium
Herbals/Botanicals—ginkgo biloba, Echinacea, garlic, black cohosh root, ginseng, St. John's Wort, kava kava, valerian, saw palmetto, soy, bilberry, green tea, milk thistle, guarana
Non-Herbals—glucosamine, chondroitin, probiotics such as lactobacillus and acidophilus, DHEA (dehydroepiandosterone), C0Q-10 (Co-Enzyme Q-10), lecithin, melatonin, flax, flaxseed oil, SAMe
Other dietary supplements—proteins such as soy protein and amino acids
Non-essential amino acids—alanine, serines, L-tyrosine, glycines, L-glutamine, L-glutamic acid, L-histidine, L-cysteine, L-aspartic acid, L-ornithine, asparagine, proline, L-arginine
Essential amino acids—threonine, L-phenylalanine, D-phenylalanine, DL-phenylalanine, L-lysine, L-leucine, L-isoleucine, L-valine, L-methionine, taurine, L-tryptophan
Functional additives—lycopene, isoflavones, tocotrienols, sterols, probiotics such as Lactobacillus acidophilus, Bifidobacterium bifidum, and Bifidobacterium longum, polyunsaturated fatty acids, fibers such as psyllium, omega-3 fatty acids such as docosahexaenoic acid (DHA) and eicosapentaenoic acid (EPA)

Many natural products, and particularly nutraceuticals, can be obtained from: Advanced Nutraceuticals, Inc. (US), Archer Daniels Midland Company (US), BASF AG (DE), Bayer AG (DE), Beaufour-Ipsen (FR), Ceapro, Inc. (CA), F. Hoffman La Roche AG (Switzerland), GlaxoSmithKline (UK), Laboratories Arkopharma SA (FR), Leiner Health Products (US), Mannatech, Inc. (US), Mead Johnson Nutritionals (US), Natrol, Inc. (US), NBTY, Inc. (US), Novartis AG (Switzerland), Nutraceutical International Corp. (US), Ocean Nutrition Canada (CA), Perrigo Company (US), Pharmavite Corp. (US), Rexall Sundown, Inc. (US), Royal Numico NV (Netherlands), Scolr Inc. (US), Twinlab Corp. (US), U.S. Nutraceuticals LLC (US), and Wyeth (US).

Exemplary products can be derived from a plant, fungus, bacteria, or animal, e.g., from Achillea millefolium, Arctium lappa, Arnica chamissonis, Artemisia absinthum, Astragalus membranaceus, Borago officinalis, Calendula officinalis, Catha edulis, Centaurea cyanoides, Cheiranthus cheiri, Chelidonium majus, Cichorium pumilum, Citrullus colocynthis, Cynara cardunculus, Echinacea angustifolia, Echinacea pallida, Echinacea purpurea, Eruca satvia, Eschscholzia californica, Filipendula ulmaria, Galega officinalis, Ginko biloba, Glechloma hederacea, Hypericum perforatum, Hypericum triquetrifolium, Hyssopus officinalis, Leonurus cardiaca, Lippia citriodora, Majorana syriaca, Marrubium valgare, Melissa officinalis, Mentha spicata, Mentha piperita, Mercurialis anizua L., Micromeria fruticosa, Nepeta cataria, Olea europaea, Origanum vulgare, Passiflora incarnata, Plantago mayor, Rosmarinus officinals, Ruta graveolens, Salvia hierosolymitana, Salvia officinalis, Salvia sclarea, Satureja hortensis, Satureja thymbra, Scutellaria baicalensis, Scutellaria laterifolia, Stellaria media, Stevia rabaudiana, Symphytum officinale, Tanacetum partheneum, Taraxacum officinale, Thymus hyb. lemon, Thymus vulgaris, Tribulus terrestis, Urtica urens, Valeriana officinalis, Verbascum sinuatum, Verbascum thapsus, Verbena officinalis, Vitex agnus-castus, and Withania somenifera.

The Centenarian Genome

The centenarian genome is different from the general population in at least two ways. It has a low occurrence of deleterious genetic variants that predispose for disease, and a high concentration of protective genetic variants that stave off disease and the effects of aging, without harmful side effects. A database can be used to identify both detrimental and protective genes and use these identified genes to customize longevity promoting supplements.

One way of identifying these “lifespan” genes is genetic association studies. Variants of the genes MTP, APOE, and CETP are examples of genes that are associated with altered lifespan.

Genetic variants affecting lifespan can be classified as either “protective” or “detrimental” risk factors for living a long life.

The MTP Locus

Longevity in humans is associated with certain genetic polymorphisms on chromosome IV. The most significantly implicated locus is linked to single nucleotide polymorphism (SNP) marker rs1553432 and corresponds to the micosomal triglyceride transfer protein (MTP) locus, e.g., a region within 80 kb of the MTP gene ATG. An exemplary MTP gene encodes an amino acid described in GenBank® reference NP_{—}000244.1. This locus is within a larger region that has linkage at the D4S1564 marker (also referred to as AFM248zg9, 248zg9, and Z23817) on human chromosome 4, and which is approximately 10-20 cM in size. This larger 10-20 cM region with linkage at the D4S1564 marker is described in PCT WO 02/14552.

Markers in the MTP locus can be used as indicators for the presence of a genetic predisposition that is manifested as increased longevity or conversely reduced susceptibility to diseases causing mortality. Particularly useful markers include, for example, rs1800591 and rs2866164, and rs1553432.

Extracts of garlic and citric flavanoids, for example, are two naturally occurring products that decrease MTP expression levels and thereby offset the impact of the detrimental variant.

TABLE 1
Risk allele frequencies

| | |
|---|---|
| −493G allele | −493T allele |

Cases (long-lived)

| | | |
|---|---|---|
| 95Q allele | 546 (76%) | 127 (17%) |
| 95H allele | 0 | 53 (7%) |

Controls

| | | |
|---|---|---|
| 95Q allele | 498 (68%) | 201 (27%) |
| 95H allele | 0 | 36 (5%) |

Table 1. Risk haplotype allele frequencies. Broken down into cases (long-lived) and controls, shows frequencies for the four possible haplotypes defined by the promoter (−493 G/T) and exon 3 (95 Q/H) polymorphisms. Note that only three of the four haplotypes was observed, fulfilling the criteria of no historic recombination between the two SNPs. 726 out of 760 case chromosomes were successfully genotyped at both alleles in the long-lived individuals, compared to 735 out of 760 for the controls. As discussed in the text, the haplotype composed of the −493T allele and 95Q allele is underrepresented in long-lived individuals, suggesting this variant confers mortality risk.

Exemplary detrimental alleles for the MTP locus include the 95H allele and −493T allele. Exemplary detrimental haplotypes for the MTP locus include the 95Q and −493T alleles, the 95H and −493T alleles, and the 95H and −493G alleles. Presence of these alleles, particularly for individuals who are homozygous, can indicate that the individual should consume a nutraceutical, e.g., an extracts of garlic, a citric flavanoids, or other compound that modulates (e.g., increases) MTP expression.

The CETP Locus

The cholesteryl ester transfer protein (CETP) locus has also been implicated in longevity. CETP is a plasma protein that mediates the exchange of cholesteryl ester in high-density lipoprotein (HDL) particles for triglycerides in very low density lipoprotein (VLDL). An exemplary CETP gene encodes an amino acid described in GenBank® reference NP_{—}000069. Typically, increased CETP activity leads to increased low density lipoprotein (LDL) and VLDL levels, with a corresponding higher risk of atherosclerosis (Marotti et al. 1993. Nature 364:73-5). Decreased CETP activity leads to increased HDL levels and a lowered risk of atherosclerosis (Okamoto et al. 2000. Nature 406:203-7).

Several polymorphisms of the CETP gene have been described, some of which are associated with both increased and decreased longevity (Brown et al. 1989. Nature 342:448-51; Arai et al. 1996. J. Lipid Res. 37:2145-54; Tamminen et al. 1996. Atherosclerosis 124:237-47; Kakko et al. 1998. Atherosclerosis 136:233-40; Okumura et al. 2002. Atherosclerosis 161:425-31; Wang et al. 2002. Clin. Chim. Acta 322:85-90; Barzilai et al. 2003. JAMA 290:2030-40; Blankenberg et al. 2003. J. Am. Coll. Cardiol. 41:1983-9; Thompson et al. 2003. Atherosclerosis 167:195-204; Klerkx et al. 2003. Hum. Mol. Genet. 12:111-23; Lira et al. 2004. Biochim. Biophys. Acta 1684:38-45)

Exemplary CETP alleles include the I405V, TaqIB, −629, and D442G alleles.

Individuals with alleles of the CETP gene that are associated with reduced longevity can be indicated for consuming a nutraceutical, e.g., a nutraceutical which modulates (e.g., increases) HDL levels or niacin.

Methods of Evaluating Genetic Parameters

There are numerous ways of evaluating genetic parameters. Nucleic acid samples can analyzed using biophysical techniques (e.g., hybridization, electrophoresis, and so forth), sequencing, enzyme-based techniques, and combinations-thereof. For example, hybridization of sample nucleic acids to nucleic acid microarrays can be used to evaluate sequences in an mRNA population and to evaluate genetic polymorphisms. Other hybridization based techniques include sequence specific primer binding (e.g., PCR or LCR); Southern analysis of DNA, e.g., genomic DNA; Northern analysis of RNA, e.g., mRNA; fluorescent probe based techniques Beaudet et al. (2001) Genome Res. 11(4):600-8; allele specific amplification. Enzymatic techniques include restriction enzyme digestion; sequencing; and single base extension (SBE). These and other techniques are well known to those skilled in the art.

Electrophoretic techniques include capillary electrophoresis and Single-Strand Conformation Polymorphism (SSCP) detection (see, e.g., Myers et al (1985) Nature 313:495-8 and Ganguly (2002) Hum Mutat. 19(4):334-42). Other biophysical methods include denaturing high pressure liquid chromatography (DHPLC).

In one embodiment, allele specific amplification technology that depends on selective PCR amplification may be used to obtain genetic information. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it is possible to introduce a restriction site in the region of the mutation to create cleavage-based detection (Gasparini et al. (1992) Mol. Cell Probes 6:1). In another embodiment, amplification can be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Enzymatic methods for detecting sequences include amplification based-methods such as the polymerase chain reaction (PCR; Saiki, et al. (1985) Science 230, 1350-1354) and ligase chain reaction (LCR; Wu. et al. (1989) Genomics 4, 560-569; Barringer et al. (1990), Gene 1989, 117-122; F. Barany. 1991, Proc. Natl. Acad. Sci. USA 1988, 189-193); transcription-based methods utilize RNA synthesis by RNA polymerases to amplify nucleic acid (U.S. Pat. No. 6,066,457; U.S. Pat. No. 6,132,997; U.S. Pat. No. 5,716,785; Sarkar et al., Science (1989) 244:331-34; Stofler et al., Science (1988) 239:491); NASBA (U.S. Pat. Nos. 5,130,238; 5,409,818; and 5,554,517); rolling circle amplification (RCA; U.S. Pat. Nos. 5,854,033 and 6,143,495) and strand displacement amplification (SDA; U.S. Pat. Nos. 5,455,166 and 5,624,825). Amplification methods can be used in combination with other techniques.

Other enzymatic techniques include sequencing using polymerases, e.g., DNA polymerases and variations thereof such as single base extension technology. See, e.g., U.S. Pat. No. 6,294,336; U.S. Pat. No. 6,013,431; and U.S. Pat. No. 5,952,174

Mass spectroscopy (e.g., MALDI-TOF mass spectroscopy) can be used to detect nucleic acid polymorphisms. In one embodiment, (e.g., the MassEXTEND™ assay, SEQUENOM, Inc.), selected nucleotide mixtures, missing at least one dNTP and including a single ddNTP is used to extend a primer that hybridizes near a polymorphism. The nucleotide mixture is selected so that the extension products between the different polymorphisms at the site create the greatest difference in molecular size. The extension reaction is placed on a plate for mass spectroscopy analysis.

Fluorescence based detection can also be used to detect nucleic acid polymorphisms. For example, different terminator ddNTPs can be labeled with different fluorescent dyes. A primer can be annealed near or immediately adjacent to a polymorphism, and the nucleotide at the polymorphic site can be detected by the type (e.g., “color”) of the fluorescent dye that is incorporated.

Hybridization to microarrays can also be used to detect polymorphisms, including SNPs. For example, a set of different oligonucleotides, with the polymorphic nucleotide at varying positions with the oligonucleotides can be positioned on a nucleic acid array. The extent of hybridization as a function of position and hybridization to oligonucleotides specific for the other allele can be used to determine whether a particular polymorphism is present. See, e.g., U.S. Pat. No. 6,066,454.

In one implementation, hybridization probes can include one or more additional mismatches to destabilize duplex formation and sensitize the assay. The mismatch may be directly adjacent to the query position, or within 10, 7, 5, 4, 3, or 2 nucleotides of the query position. Hybridization probes can also be selected to have a particular Tₘ, e.g., between 45-60° C., 55-65° C., or 60-75° C. In a multiplex assay, Tₘ's can be selected to be within 5, 3, or 2° C. of each other.

It is also possible to directly sequence the nucleic acid for a particular genetic locus, e.g., by amplification and sequencing, or amplification, cloning and sequence. High throughput automated (e.g., capillary or microchip based) sequencing apparati can be used. In still other embodiments, the sequence of a protein of interest is analyzed to infer its genetic sequence. Methods of analyzing a protein sequence include protein sequencing, mass spectroscopy, sequence specific immunoglobulins, and protease digestion.

Another exemplary profile is a function of an evaluation of gene expression, e.g., using a microarray. Accordingly, in some embodiments, transcripts are analyzed from a subject. One method for comparing transcripts uses nucleic acid microarrays that include a plurality of addresses, each address having a probe specific for a particular transcript. Such arrays can include at least 100, or 1000, or 5000 different probes, so that a substantial fraction, e.g., at least 10, 25, 50, or 75% of the genes in an organism are evaluated. mRNA can be isolated from a sample of the organism or the whole organism. The mRNA can be reversed transcribed into labeled cDNA. The labeled cDNAs are hybridized to the nucleic acid microarrays. The arrays are detected to quantitate the amount of cDNA that hybridizes to each probe, thus providing information about the level of each transcript.

Methods for making and using nucleic acid microarrays are well known. For example, nucleic acid arrays can be fabricated by a variety of methods, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin based methods (e.g., as described in U.S. Pat. No. 5,288,514), and bead based techniques (e.g., as described in PCT US/93/04145). The capture probe can be a single-stranded nucleic acid, a double-stranded nucleic acid (e.g., which is denatured prior to or during hybridization), or a nucleic acid having a single-stranded region and a double-stranded region. Preferably, the capture probe is single-stranded. The capture probe can be selected by a variety of criteria, and preferably is designed by a computer program with optimization parameters. The capture probe can be selected to hybridize to a sequence rich (e.g., non-homopolymeric) region of the nucleic acid. The Tₘ of the capture probe can be optimized by prudent selection of the complementarity region and length. Ideally, the Tₘ of all capture probes on the array is similar, e.g., within 20, 10, 5, 3, or 2° C. of one another. A database scan of available sequence information for a species can be used to determine potential cross-hybridization and specificity problems.

The isolated mRNA from samples for comparison can be reversed transcribed and optionally amplified, e.g., by rtPCR, e.g., as described in (U.S. Pat. No. 4,683,202). The nucleic acid can be labeled during amplification, e.g., by the incorporation of a labeled nucleotide. Examples of preferred labels include fluorescent labels, e.g., red-fluorescent dye Cy5 (Amersham) or green-fluorescent dye Cy3 (Amersham), and chemiluminescent labels, e.g., as described in U.S. Pat. No. 4,277,437. Alternatively, the nucleic acid can be labeled with biotin, and detected after hybridization with labeled streptavidin, e.g., streptavidin-phycoerythrin (Molecular Probes).

The labeled nucleic acid can be contacted to the array. In addition, a control nucleic acid or a reference nucleic acid can be contacted to the same array. The control nucleic acid or reference nucleic acid can be labeled with a label other than the sample nucleic acid, e.g., one with a different emission maximum. Labeled nucleic acids can be contacted to an array under hybridization conditions. The array can be washed, and then imaged to detect fluorescence at each address of the array.

A general scheme for producing and evaluating profiles can include the following. The extent of hybridization at an address is represented by a numerical value and stored, e.g., in a vector, a one-dimensional matrix, or one-dimensional array. The vector x has a value for each address of the array. For example, a numerical value for the extent of hybridization at a first address is stored in variable xₐ. The numerical value can be adjusted, e.g., for local background levels, sample amount, and other variations. Nucleic acid is also prepared from a reference sample and hybridized to an array (e.g., the same or a different array), e.g., with multiple addresses. The vector y is construct identically to vector x. The sample expression profile and the reference profile can be compared, e.g., using a mathematical equation that is a function of the two vectors. The comparison can be evaluated as a scalar value, e.g., a score representing similarity of the two profiles. Either or both vectors can be transformed by a matrix in order to add weighting values to different nucleic acids detected by the array.

The expression data can be stored in a database, e.g., a relational database such as a SQL database (e.g., Oracle or Sybase database environments). The database can have multiple tables. For example, raw expression data can be stored in one table, wherein each column corresponds to a nucleic acid being assayed, e.g., an address or an array, and each row corresponds to a sample. A separate table can store identifiers and sample information, e.g., the batch number of the array used, date, and other quality control information.

Other methods for quantitating nucleic acid species include: quantitative RT-PCR. In addition, two nucleic acid populations can be compared at the molecular level, e.g., using subtractive hybridization or differential display.

In addition, once a set of nucleic acid transcripts are identified as being associated with long lived individuals or centenarians, it is also possible to develop a set of probes or primers that can evaluate a sample for such markers. For example, a nucleic acid array can be synthesized that includes probes for each of the identified markers.

Protein Analysis

The abundance of a plurality of protein species can be determined in parallel, e.g., using an array format, e.g., using an array of antibodies, each specific for one of the protein species. Other ligands can also be used. Antibodies specific for a polypeptide can be generated by known methods.

Methods for producing polypeptide arrays are described, e.g., in De Wildt et al., (2000) Nature Biotech. 18:989-994; Lueking et al., (1999) Anal. Biochem. 270:103-111; Ge, H. (2000) Nucleic Acids Res. 28:e3, I-VII; MacBeath and Schreiber, (2000) Science 289, 1760-1763; Haab et al., (2001) Genome Biology 2(2):research0004.1; and WO 99/51773A1. A low-density (96 well format) protein array has been developed in which proteins are spotted onto a nitrocellulose membrane Ge, H. (2000) Nucleic Acids Res. 28, e3, I-VII). A high-density protein array (100,000 samples within 222×222 mm) used for antibody screening was formed by spotting proteins onto polyvinylidene difluoride (PVDF) (Lueking et al. (1999) Anal. Biochem. 270, 103-111). Polypeptides can be printed on a flat glass plate that contained wells formed by an enclosing hydrophobic Teflon mask (Mendoza, et al. (1999). Biotechniques 27, 778-788.). Also, polypeptide can be covalently linked to chemically derivatized flat glass slides in a high-density array (1600 spots per square centimeter) (MacBeath, G., and Schreiber, S. L. (2000) Science 289, 1760-1763). De Wildt et al., describe a high-density array of 18,342 bacterial clones, each expressing a different single-chain antibody, in order to screening antibody-antigen interactions (De Wildt et al. (2000). Nature Biotech. 18, 989-994). These art-known methods and other can be used to generate an array of antibodies for detecting the abundance of polypeptides in a sample. The sample can be labeled, e.g., biotinylated, for subsequent detection with streptavidin coupled to a fluorescent label. The array can then be scanned to measure binding at each address and analyze similar to nucleic acid arrays.

Mass Spectroscopy.

Mass spectroscopy can also be used, either independently or in conjunction with a protein array or 2D gel electrophoresis. For 2D gel analysis, purified protein samples from the first and second organism are separated on 2D gels (by isoelectric point and molecular weight). The gel images can be compared after staining or detection of the protein components. Then individual “spots” can be proteolyzed (e.g., with a substrate-specific protease, e.g., an endoprotease such as trypsin, chymotrypsin, or elastase) and then subjected to MALDI-TOF mass spectroscopy analysis. The combination of peptide fragments observed at each address can be compared with the fragments expected for an unmodified protein based on the sequence of nucleic acid deposited at the same address. The use of computer programs (e.g., PAWS) to predict trypsin fragments, for example, is routine in the art. Thus, each address of spot on a gel or each address on a protein array can be analyzed by MALDI. The data from this analysis can be used to determine the presence, abundance, and often the modification state of protein biomolecules in the original sample. Most modifications to proteins cause a predictable change in molecular weight.

Other Methods.

Other methods can also be used to profile the properties of a plurality of protein biomolecules. These include ELISAs and Western blots. Many of these methods can also be used in conjunction with chromatographic methods and in situ detection methods (e.g., to detect subcellular localization).

Molecular Biology Techniques

Methods described herein can include use of routine techniques in the field of molecular biology, biochemistry, classical genetics, and recombinant genetics. Basic texts disclosing the general methods of use in this invention include Sambrook & Russell, Molecular Cloning: A Laboratory Manual, 3rd Edition, Cold Spring Harbor Laboratory, N.Y. (2001); Kriegler, Gene Transfer and Expression: A Laboratory Manual (1990); Scopes (1994) Protein Purification: Principles and Practice, New York: Springer-Verlag; and Ausubel et al., Current Protocols in Molecular Biology (Greene Publishing Associates and Wiley Interscience, N.Y. (1989).

Other Biomolecules

Another exemplary profile includes one or more values that are a function of an evaluation of another biological factor, e.g., a metabolite. Other biomolecules (e.g., other than proteins and nucleic acids, e.g., metabolites, such as sugars) can be detected by a variety of methods include: ELISA, antibody binding, mass spectroscopy, enzymatic assays, chemical detection assays, and so forth.

Any combination of the above methods can also be used. For example, a profile can include one parameter that is associated with a genomic component (e.g., a SNP) and another that relates to gene expression. The above methods can be used to evaluate a genetic position, e.g., a chromosomal position (e.g., a SNP) associated with a protective or detrimental effect on aging, expression of a gene associated with a protective or detrimental effect on aging, or a factor associated with a protective or detrimental effect on aging (e.g., cholesterol, or a particle such as HDL, LDL, etc.).

Computer Implementations

Aspects of the invention can be implemented in digital electronic circuitry, or in computer hardware, firmware, software, or in combinations thereof. Methods of the invention can be implemented using a computer program product tangibly embodied in a machine-readable storage device for execution by a programmable processor; and method actions can be performed by a programmable processor executing a program of instructions to perform functions of the invention by operating on input data and generating output. For example, the invention can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object oriented programming language, or in assembly or machine language if desired; and in any case, the language can be a compiled or interpreted language. Suitable processors include, by way of example, both general and special purpose microprocessors. A processor can receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks such as, internal hard disks and removable disks; magneto-optical disks; and CD_ROM disks. Any of the foregoing can be supplemented by, or incorporated in, ASICs (application-specific integrated circuits).

An exemplary system includes a processor, a random access memory (RAM) , a program memory (for example, a writable read-only memory (ROM) such as a flash ROM), a hard drive controller, and an input/output (I/O) controller coupled by a processor (CPU) bus. The system can be preprogrammed, in ROM, for example, or it can be programmed (and reprogrammed) by loading a program from another source (for example, from a floppy disk, a CD-ROM, or another computer).

The hard drive controller is coupled to a hard disk suitable for storing executable computer programs, including programs embodying the present invention, and data including storage. The I/O controller is coupled by means of an I/O bus to an I/O interface. The I/O interface receives and transmits data in analog or digital form over communication links such as a serial link, local area network, wireless link, and parallel link.

One non-limiting example of an execution environment includes computers running Linux Red Hat OS, Windows XP (Microsoft), Windows NT 4.0 (Microsoft) or better or Solaris 2.6 or better (Sun Microsystems) operating systems. Browsers can be Microsoft Internet Explorer version 4.0 or greater or Netscape Navigator or Communicator version 4.0 or greater. Computers for databases and administration servers can include Windows NT 4.0 with a 400 MHz Pentium II (Intel) processor or equivalent using 256 MB memory and 9 GB SCSI drive. For example, a Solaris 2.6 Ultra 10 (400Mhz) with 256 MB memory and 9 GB SCSI drive can be used. Other environments can also be used.

A number of embodiments of the invention have been described. Nevertheless, it will be understood that various modifications may be made without departing from the spirit and scope of the invention. Accordingly, other embodiments are within the scope of the following claims.

What is claimed is:

1. A method of identifying an association between a composition and a biological parameter, the method comprising
contacting the composition to a test cell or administering the composition to a test organism;
evaluating the test cell or test organism to determine a profile that comprises one or more parameters;
comparing the profile to a database of profile to obtain an evaluation of whether the composition modulates a lifespan-extending trait.

2. The method of claim 1 wherein the composition comprises a nutraceutical or natural product.

3. A method of improving health of an individual, the method comprising:
evaluating a longevity-associated gene of a subject; and
if the subject has a detrimental allele of the gene, providing, to the subject, a natural product that modulates expression or activity of the gene or its gene product.

4. The method of claim 3 wherein the subject is a human.

5. The method of claim 3 wherein the gene is MTP or CETP.

6. A method of improving health of an individual, the method comprising:
administering a nutraceutical to a subject who has a detrimental allele of MTP, wherein the nutraceutical increases expression of the MTP gene.

7. The method of claim 6 wherein the nutraceutical comprises a garlic extract or a citric flavenoid.

8. A method of improving health of an individual, the method comprising:
administering a nutraceutical to a subject who has a detrimental allele of CETP, wherein the nutraceutical increases HDL levels.

9. The method of claim 8 wherein the nutraceutical comprises a niacin.

10. A method comprising:
determining a biological parameter of a subject;
comparing the parameter to a database of parameters of protective individuals and associated natural products; and
recommending a natural product based on a comparison of the parameter for the subject and information in the database.

11. The method of claim 10, wherein the parameter relates to expression or activity of a gene or protein.

12. The method of claim 10, wherein the parameter relates to a protein modification or protein localization.

13. The method of claim 10, wherein the parameter relates to the MTP or CETP gene or gene product.

14. The method of claim 10, wherein the comparison is made using profiles that each comprise a plurality of parameters.

15. The method of claim 10, wherein the comparison is made using a distance function.

16. The method of claim 10, wherein the recommendations are delivered across a network.

17. The method of claim 10, wherein the recommendations are accessible through a web site.

18. The method of claim 10, wherein the subject is monitored before, during, or after administering the recommended natural product.

19. A method of identifying an association between a test compound and a biological parameter, the method comprising:
contacting the composition to a test cell that is not a protective cell or an organism that is not a protective individual;
determining a biological parameter of the test cell or organism;
evaluating if the composition alters a parameter of the test organism to provide a protective effect characteristic of parameters of protective individuals.

20. The method of claim 19, wherein the composition is a nutraceutical.

21. The method of claim 19, wherein the composition is a diet or dietary substance.

22. The method of claim 19, wherein the test cell is derived from an organism that does not have a lifespan-extending trait, or the test organism does not have a life-span extending trait.

23. The method of claim 22, wherein the trait is an allele of the MTP gene.

24. The method of claim 22, wherein the trait is an allele of the CETP gene.

25. A method of identifying a composition, the method comprising:
providing a desirable profile from a protective individual or materials derived therefrom;
screening test compositions using a non-protective individual or materials derived therefrom;
selecting a composition that changes the profile of the non-protective individual or materials derived therefrom so that it becomes more similar to that of the protective individual.
